(19) United States
(12) Patent Application Publication
(10) Pub. No.: US 2022/0320833 A1
(43) Pub. Date: Oct. 6, 2022

(54) Ionic Air Flow Generator, With Emitter And Collector Stripes

(71) Applicant: Ventiva, Inc., Fremont, CA (US)

(72) Inventors: Gary Alfred Oliverio, San Jose, CA (US); Carl Paul Schlachte, Ben Lomond, CA (US); Himanshu Pokharna, Saratoga, CA (US)

(21) Appl. No.: 17/735,084

(22) Filed: May 2, 2022

Related U.S. Application Data
(63) Continuation of application No. PCT/US2022/022311, filed on Mar. 29, 2022.
(60) Provisional application No. 63/168,192, filed on Mar. 30, 2021.

(51) Int. Cl. H01T 23/00

(52) U.S. Cl. CPC H01T 23/00

(57) ABSTRACT

Emitter wires and collector pins of current ionic air flow generator designs are replaced by conductors joined to a dielectric substrate, such as metal deposited on the dielectric substrate. One conductor, which is shaped to form the emitter with sharp edges, is joined to one side of the dielectric substrate. Another conductor, which is shaped to form the collector with rounded edges, is joined to the opposite side of the dielectric substrate. The dielectric substrate is not solid. It is shaped with voids that form an air gap between the emitter and the collector. Thus, when a voltage is applied to the emitter, air is ionized at the emitter. The ionized air is drawn electrostatically to the lower-voltage collector, which, through collision with neutral molecules that in turn impart their momentum, creates a flow of air through the air gap.

### CROSS-REFERENCE TO RELATED APPLICATION(S)

This application is a continuation of International Application No. PCT/US22/022311, “Ionic Air Flow Generator,” filed Mar. 29, 2022; which claims priority to U.S. Provisional Patent Application Ser. No. 63/168,192, “Ionic Air Flow Generator,” filed Mar. 30, 2021. The subject matter of all of the foregoing is incorporated herein by reference in their entirety.

### BACKGROUND

### 1. Technical Field

This disclosure relates generally to ionic air flow generators.

### 2. Description of Related Art

Many designs of ionic air flow generators use suspended lengths of wires to form the emitter and pins or a perforated plate to form the collector. However, these designs may have the following drawbacks. There are limits on how small these devices can be made. A wire may be used as the emitter, which can introduce weak points. The wire typically must be attached to a substrate or frame. The attachment may be difficult or a weak point. It can be difficult to keep the wire under proper tension, which is necessary to maintain consistent emitter-collector spacing and co-planarity between the emitter and collector, among other requirements, and the collector construction may also be subject to warpage or imperfections which can lead to non-planar geometry. The substrate or frame also adds to the size of the device.

Thus, there is a need for better approaches to ionic air flow generators.

### BRIEF DESCRIPTION OF THE DRAWINGS

Embodiments of the disclosure have other advantages and features which will be more readily apparent from the following detailed description and the appended claims, when taken in conjunction with the examples in the accompanying drawings, in which:

FIG. 1 is a graph plotting flow rate as a function of area for air flow generators according to various embodiments described herein.

FIG. 2A is a perspective view of a unit cell used to construct an ionic air flow generator.

FIGS. 2B and 2C are plan, side and detail views of the emitter conductor and collector conductor, respectively, from FIG. 2A.

FIGS. 2D and 2E are perspective views of additional examples of units cells.

FIG. 3A is a perspective view of an assembly during manufacture of an ionic air flow generator, using the unit cell of FIGS. 2A-2C.

FIGS. 3B and 3C are plan views of the emitter conductor and collector conductor, respectively, from FIG. 3A, in the format of an example 5×5 array of cells.

FIG. 3D is a plan view of the dielectric substrate after removal of dielectric.

FIG. 4 is a side view of a spacer that increases creep distance between conductors.

FIG. 5 are plan views of an emitter conductor, dielectric substrate, and collector conductor, respectively, for an ionic air flow generator using a one-dimensional array of cells.

FIGS. 6A and 6B are a perspective view and cross-sectional view, respectively, of another ionic air flow generator based on the cell structure depicted in FIG. 2E.

FIG. 7A are plan views of a collector conductor, dielectric substrate, and emitter conductor, respectively, for an ionic air flow generator.

FIG. 7B is a plan view illustrating different creep distance paths.

FIGS. 8A and 8B are plan views of two different emitter subassemblies.

FIG. 8C is a plan view of a collector subassembly.

FIG. 8D is a cross-sectional view of an air flow generator constructed from the subassembly of FIG. 8C mounted on the subassembly of FIG. 8B.

FIGS. 9A and 9B are a perspective view and cross-sectional view, respectively, of yet another ionic air flow generator.

### DETAILED DESCRIPTION OF THE PREFERRED EMBODIMENTS

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

In one aspect, the emitter and/or collector of an ionic air flow generator are formed by conductors joined to a dielectric substrate, such as by metal deposited on a glass or ceramic substrate. One conductor, which is shaped to form the high-voltage emitter with sharp edges or other features to concentrate charge, is joined to one side of the dielectric substrate. Another conductor, which is shaped to form the low-voltage collector with rounded edges that reduce field concentration, is joined to the opposite side of the dielectric substrate. The dielectric substrate is not solid between the emitter and collector. It is shaped with voids that form an air gap between the emitter and collector. Thus, when a voltage is applied to the emitter, air is ionized at the emitter. The ionized air is drawn electrostatically to the lower-voltage collector, which, through collision with neutral molecules that in turn impart their momentum, creates a flow of air through the air gap. This approach may be used with either positive or negative corona devices.

For example, the dielectric substrate may start as a solid piece of glass or ceramic substrate. The surfaces of the substrate may be etched, scored or otherwise pre-conditioned. Conductors are deposited on opposite sides of the substrate. The surface shape of the substrate may be used to form structures in the conductors, such as sharp edges for the emitter or rounded edges for the collector. Dielectric between the conductors is removed, creating an air gap for air flow.

In one approach, sharp-edged groove(s) are made in one side of the substrate. Depositing the conductor into the grooves then forms ridges in the conductor, which functions as the emitter. Conductor is also deposited on the other side of the substrate and patterned using standard lithography processes, thus forming the collector. After the conductors are deposited, substrate material between the conductors may be removed to create a path for air flow between the emitter and collector.

In a different approach, smooth, concave grooves are made in the substrate, and depositing the conductor into the groove then forms rounded surfaces in the conductor, which functions as the collector. Conductor is also applied to the opposite side with standard lithography techniques and shaped to form sharp edges, such as from a square cross section. This then functions as the emitter. After the conductors are deposited, substrate material between the conductors may be removed to create a path for air flow between the emitter and collector.

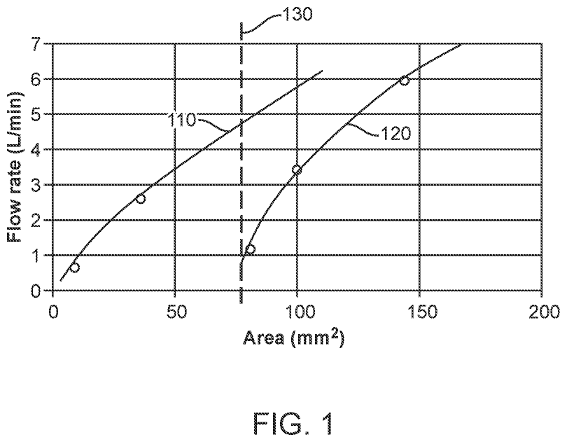

This approach allows the construction of smaller air flow generators compared to other techniques. FIG. 1 is a graph plotting flow rate as a function of cross-sectional flow area for ionic air flow generators. Curve 110 shows the flow rate achievable by an example ionic air flow generator constructed as described herein. Curve 120 shows a flow rate achievable by a typical micro axial fan. Curve 120 does not go below an area of approximately 75 mm², which is marked by the dashed line 130, because it is difficult to construct micro axial fans that are so small. In contrast, curve 110 shows that smaller size devices are possible with the approach described herein, for example flow areas of 50 mm² or even 15 mm² or less. The air gap between emitter and collector may be 2 mm or less or even 1 mm or less. The total thickness of the device may also be 5 mm or less or even 2 mm or 1 mm or less. These devices may achieve flow rates of 2, 3, or 4 liters per minute per cm² of flow area or more. These types of devices may provide active cooling of 2.5 watts using a device that is 6 mm×6 mm×2 mm.

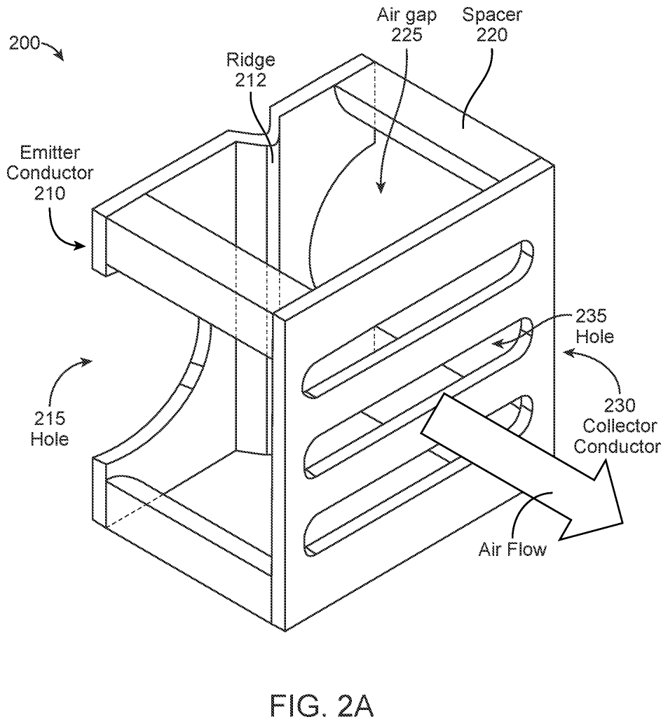

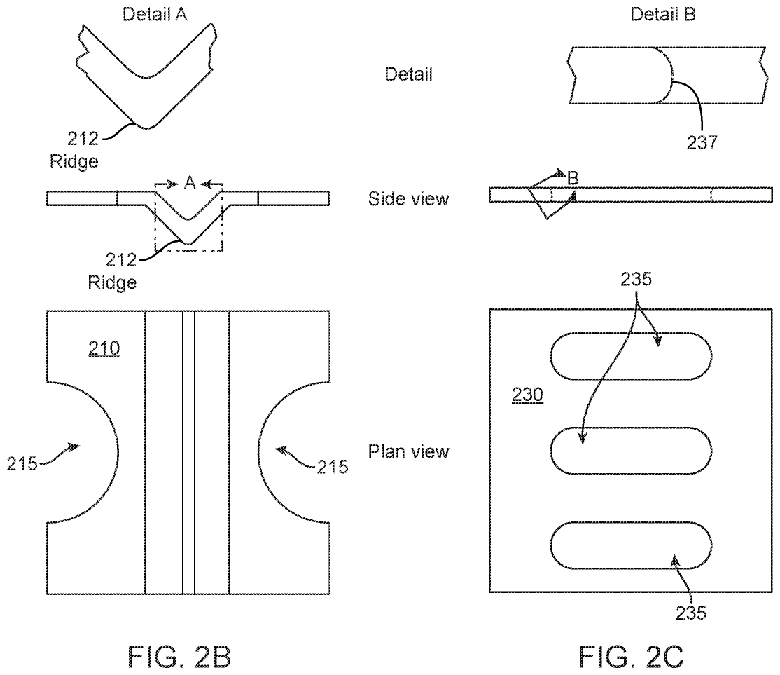

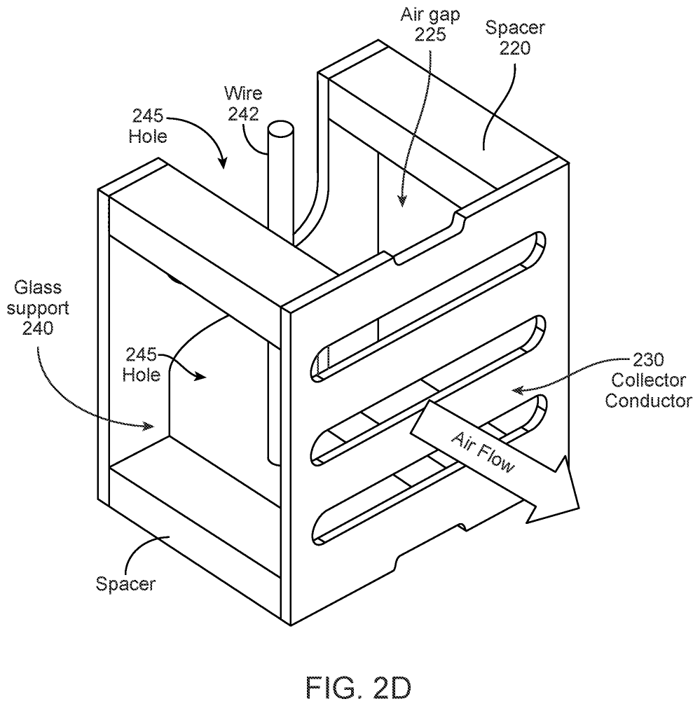

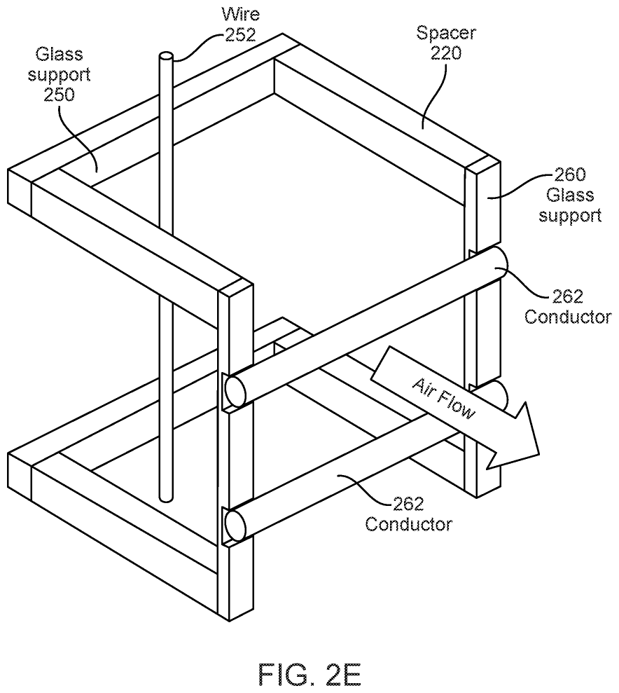

FIGS. 2-5 show one class of designs for these ionic air flow generators. FIG. 2A is a perspective view of a unit cell 200 used to construct the air flow generator. In this example, the unit cell has an area of 1 mm×1 mm, and a thickness of slightly less than 1 mm. The unit cell 200 includes two conductors 210 and 230, separated by a dielectric substrate which takes the form of spacers 220 in the final device. During construction, the two conductors 210, 230 are deposited onto a solid dielectric substrate, such as a glass or ceramic substrate. Dielectric is removed to create an air gap 225 between the two conductors 210, 230. The conductors 210, 230 include an emitter and collector, respectively. Some of the dielectric substrate remains to form the spacers 220, which maintains a consistent spacing for the air 225 gap between the emitter and collector.

FIG. 2B shows plan, side and detail views of conductor 210. Conductor 210 is predominantly flat. The flat surface areas in the corners of this unit cell for conductor 210 are joined to the spacers 220. The conductor 210 is also shaped to function as an emitter. It typically includes features that concentrate charge, such as points or edges. In this example, the conductor 210 is formed with a ridge 212 that has a sharp edge, which functions as the emitter. The radius of curvature of the ridge preferably should be as tight as possible, and preferably not larger than 30 um. This example uses a line-plane geometry. Other types of linear raised structures may also be used. If the emitter were formed as raised point structures (such as cones or pyramids), rather than raised linear structures (such as ridges), that would implement a point-plane geometry. Raised point structures preferably should also have feature sizes and curvature radii not larger than 30 um. Conductor 210 also includes holes 215 to allow air flow.

FIG. 2C shows plan, side and detail views of conductor 230. Conductor 230 is also predominantly flat and the flat surface areas in the corners of this unit cell of conductor 230 are joined to the spacers 220. The conductor 230 is also shaped to form a collector, typically avoiding features with points or edges. It also includes holes 235 to allow air flow. The holes 235 are designed to avoid corners and edges. In the plan view, the holes 235 are pill-shaped with rounded ends, rather than rectangular with corners. In the side view, the edges 237 of the holes are also rounded, particularly the edges on the side facing the emitter. Preferably, they have less curvature than the emitter ridge. This reduces the risk of unwanted arcing or breakdown.

FIGS. 2A-2C show one example of a unit cell, but other designs are also possible. In FIG. 2D, the unit cell includes a glass substrate 240 with support points for a wire 242 that functions as the emitter. The wire may have a diameter of 10-30 um, so that it is concentrates charge. Air flow occurs through holes 245.

FIG. 2E shows another unit cell. This example is similar to FIG. 2D, but the collector is also based on wires or other conductors supported periodically by dielectric. Glass or dielectric supports 250 provide support points for wire 252 that functions as the emitter. Glass or dielectric supports 260 provide support points for conductors 262 that functions as the collector. Conductors 262 are more rounded (less sharp-edged) than wire 252. For example, wire 252 may have a diameter of 10-30 um, whereas conductor 262 may have a radius of curvature of 100 um or more (i.e., diameter of 200 μm or more). The two structures are separated by spacer 220.

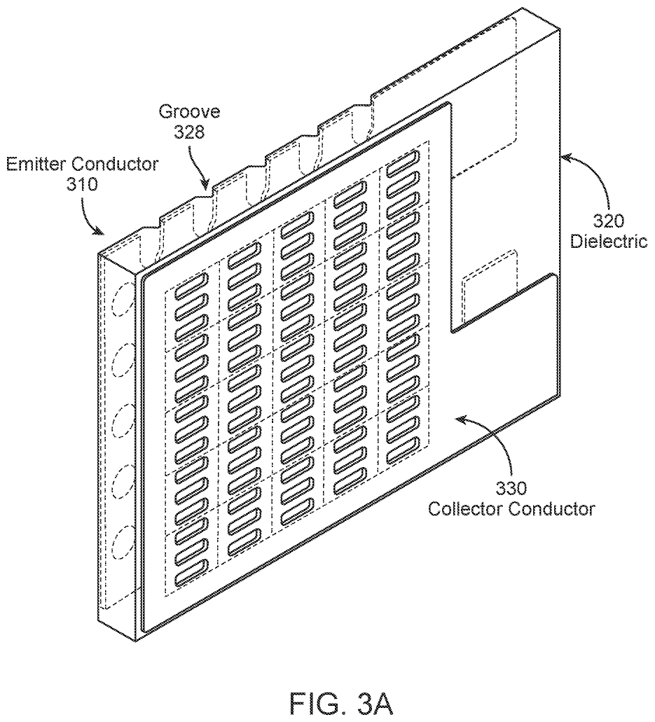

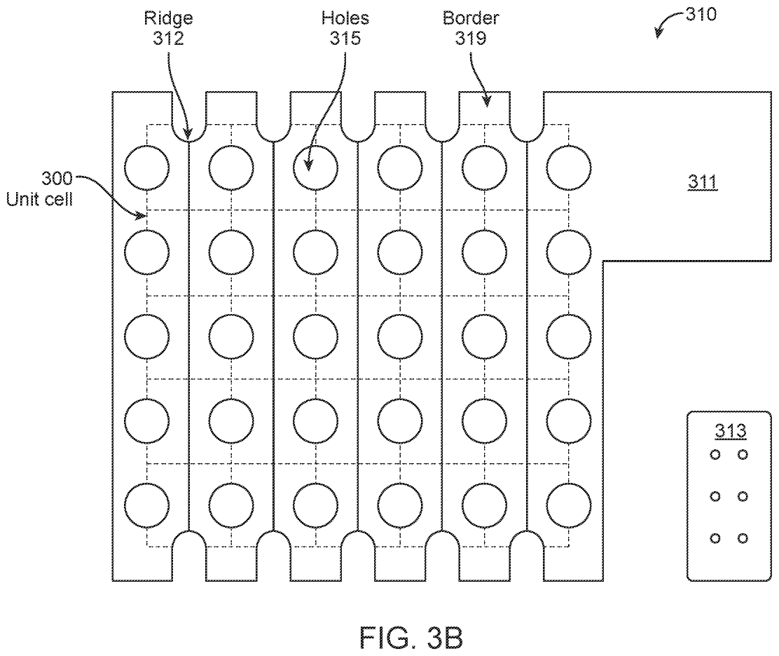

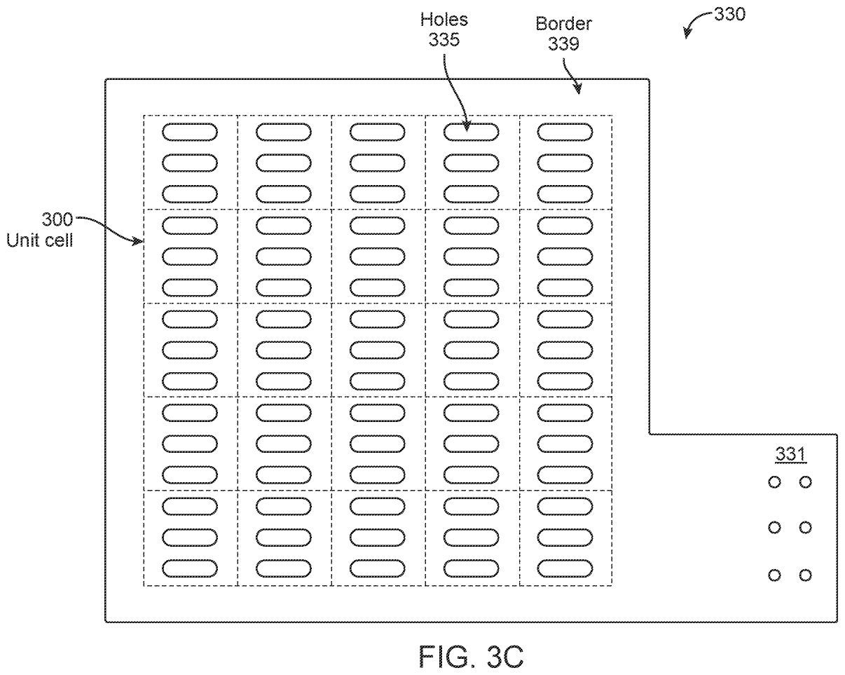

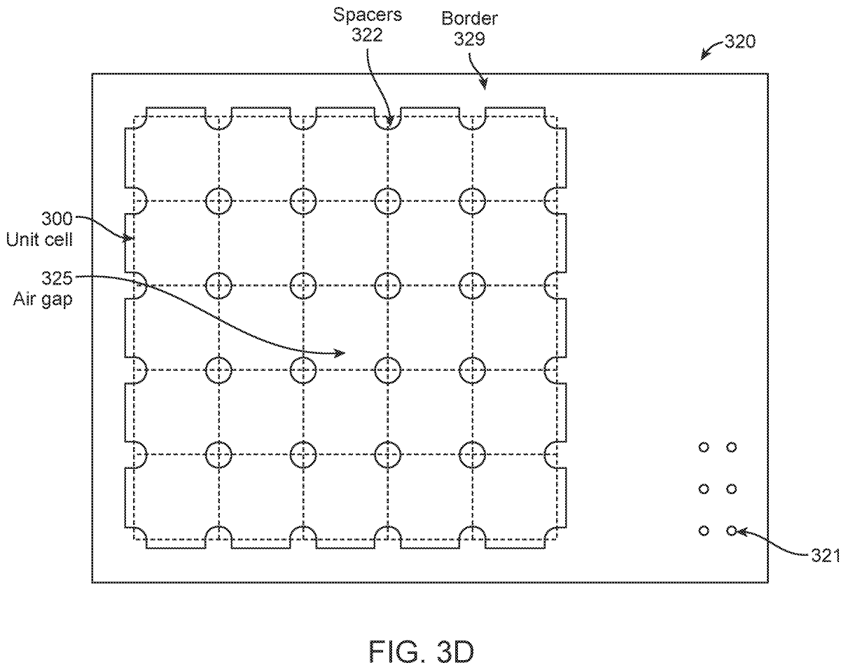

FIGS. 3A-3C show one manufacturing approach, using the unit cell of FIGS. 2A-2C. In FIG. 3A, the dielectric substrate 320 is shown before the air gap is formed. It is a solid dielectric substrate, possibly with some surface preparation. The two conductors 310, 330 are deposited onto the dielectric substrate 320 and shaped to embody specific forms, including holes, points and/or ridges. For example, grooves 328 may be scored into the dielectric 320, so that conductor 310 forms ridges. The holes in the conductors 310, 330 may be formed by etching.

FIGS. 3B and 3C show plan views of the two conductors 310, 330. In FIG. 3B, the emitter conductor 310 is constructed using a 5×5 array of the unit cell 300, as shown by the dashed lines. The emitter conductor 310 includes ridges 312 and holes 315 for air flow. The border area 319 outside the unit cells 300 may be used to join the conductor 310 to the underlying dielectric, in addition to the attachment at the spacers. FIG. 3B also shows a contact pad 311 for electrical connection to the emitter. Contact 313 is a pad that is connected to the collector conductor, so that electrical connections to both the emitter and collector may be made from one side of the device.

In FIG. 3C, the plan view of the collector conductor 330 shows holes 335 for air flow. The flat conductor area serves as the collector. Border area 330 may be used to join the conductor to the underlying dielectric. The unit cell 300 is shown by the dashed line. Contact area 331 connects to contact pad 313 on the other side of the dielectric.

The unit cell 300 is tiled in an array to form the complete device. Different numbers and arrangements of cells may form devices of different shapes and sizes. The flat border area remains joined to the dielectric substrate even after the interior substrate material is partially or fully removed. Contact areas 311 and 313 on the emitter side connect to the high voltage power supply. In plan view, the holes have curved shapes without corners. This reduces the risk of unwanted air breakdown along the surface of the insulator materials. Preferably, the holes occupy at least 40% or at least 50% of a cross-sectional flow area. In an alternate design, the emitter conductor may not have holes. Rather, the dielectric may have openings in the border to allow air to enter the device through the side. The air then flows out through the holes in the collector conductor.

FIG. 3D shows the dielectric substrate 320 after the interior dielectric has been removed. In one approach, the dielectric may be prepared, for example by forming grooves in its surface. Metal is applied to both sides of the dielectric 320. The metal is patterned by etching. Laser-assisted etching is used at elevated temperatures to remove glass from between the two metal layers, leaving the structure shown in FIG. 3D. Other processes may also be used.

Some dielectric may remain in the interior to function as spacers 322 in order to maintain consistent spacing for the air gap 325 between the emitter and collector. In this example, the dielectric forms a border 329 that completely encloses the air flow area. The dielectric may also extend beyond the edge of the conductors, so that the distance along the outer surface of the dielectric between the edges of the two conductors, known as the creep distance, is long enough to prevent undesired current flow. FIG. 3D also shows the vias 321 that connect electrodes 313 and 331.

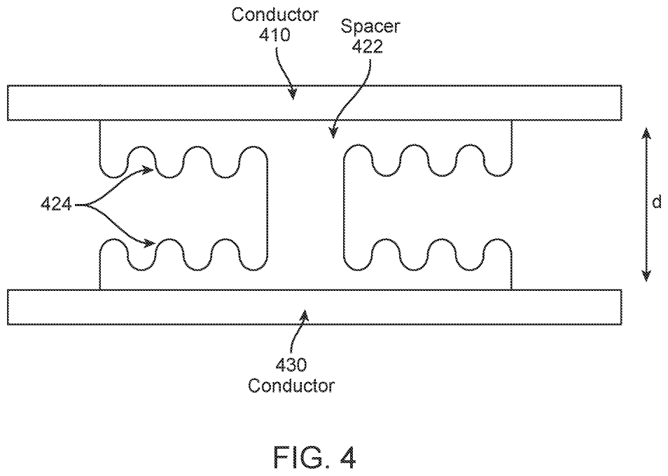

FIG. 4 is a side view of a design for a spacer 422 that increases the creep distance, thus preventing undesired electrical air breakdown along the edge of the spacer. In FIG. 4, the spacer is not just a cylindrical pillar as depicted previously. Instead, it has shape features 424 that increase the shortest distance between conductor 410 and conductor 430 along the surface of the dielectric. Without these features, the creep distance would be d, the direct distance between the two conductors. It is preferable that the creep distance exceed the direct distance between closest elements of the emitter and collector by a multiple of at least 2 or 3 or greater.

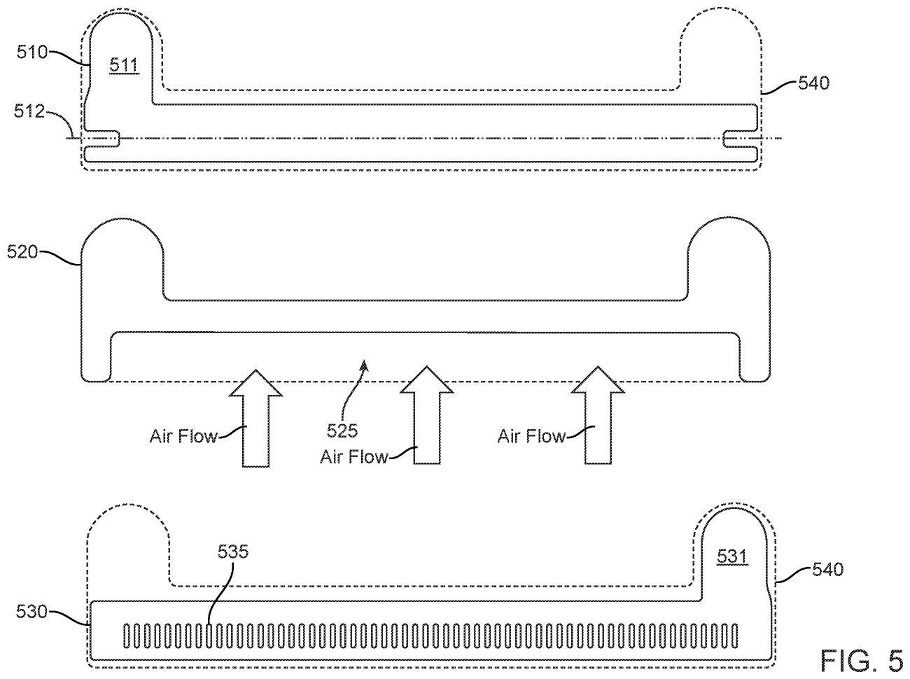

FIG. 5 are plan views of an emitter conductor 510, dielectric substrate 520 and collector conductor 530, respectively, for an ionic air flow generator using a one-dimensional array of cells. For the emitter 510, the location of the ridge line is shown by dashed line 512. Contact area 511 provides electrical connection to the conductor. There are no holes in the conductor 510. In this design, air does not flow through conductor 510. Rather, the air gap 525 in dielectric 520 has an open side and air flows through this side opening, as indicated by the arrows. Collector 530 includes holes 535 to allow air flow (in the direction out of the paper in FIG. 5). Electrode 531 provides electrical connection. The dashed shape 540 shows the footprint of the device.

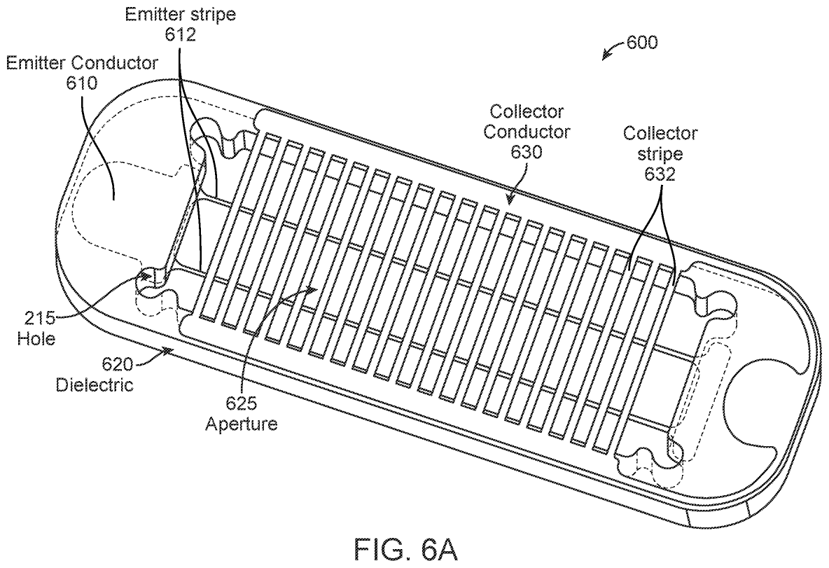

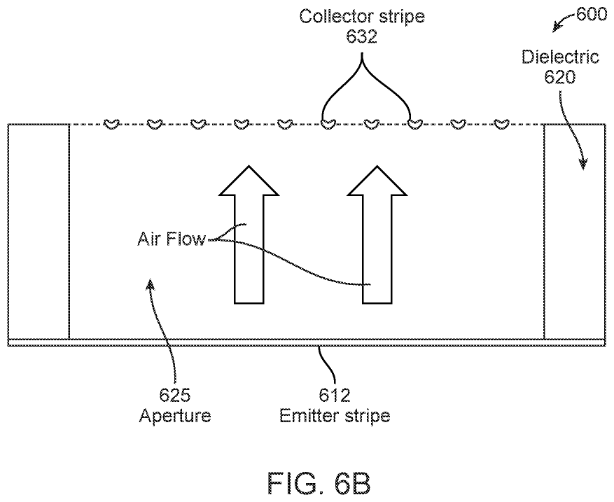

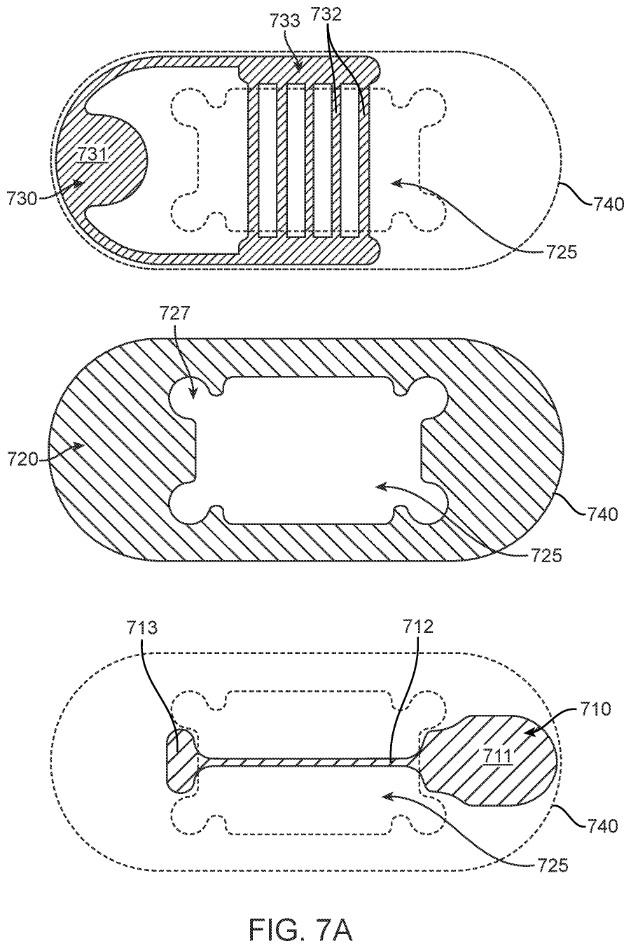

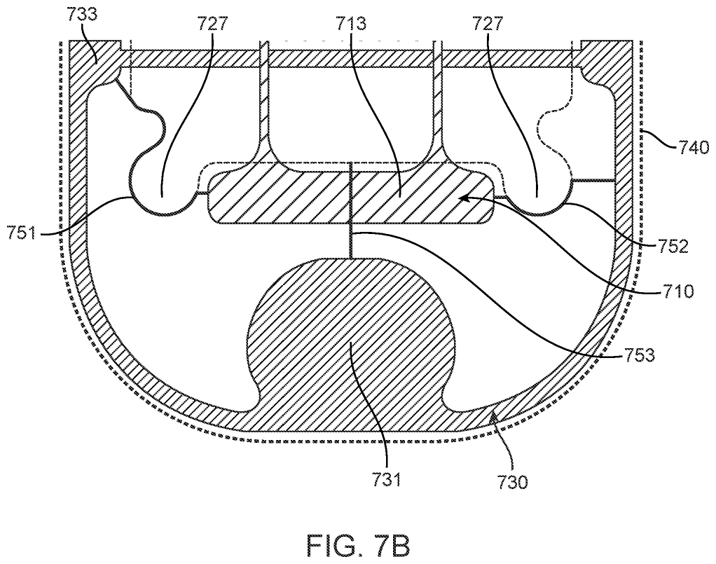

FIGS. 6-7 show another class of designs for ionic air flow generators. FIG. 6A is a perspective view of an example of this class of ionic air flow generator 600. Here, the device 600 includes two conductors 610 and 630, separated by a dielectric 620. During construction, the two conductors 610, 630 are deposited onto a solid dielectric substrate, such as a glass or ceramic substrate. Dielectric is removed to create an aperture 625 in the dielectric substrate. Conductor 610 includes an emitter with one or more emitter stripes 612 suspended across the aperture 625. In this example, there are two emitter stripes. Conductor 630 includes a collector with multiple collector stripes 632, also suspended across the aperture 625.

In this example, both the emitter stripes 612 and the collector stripes 632 are supported by the dielectric 620 only on the two ends of the stripes after the dielectric material has been removed. There are no mid-stripe supports. However, the length of the stripes is short enough that there is no appreciable sag, and the dielectric 620 maintains a consistent spacing for the air gap 625 between the emitter stripes 612 and collector stripes 632. In this design, the emitter stripes and collector stripes are arranged in a regular pattern, and they are oriented perpendicular to each other.

FIG. 6B is a cross-sectional view of the device, taken along one of the emitter stripes (i.e., in a plane perpendicular to the collector stripes). For convenience, FIG. 6B shows only a few collector stripes 632. The collector stripes 632 are rounded to avoid concentrating the electric field. In one approach, they are fabricated by scoring rounded grooves into the substrate (rather than the sharp-edged grooves in the previous examples of FIGS. 2-5). Metal is applied to both sides of the dielectric 620. The metal deposited into the rounded grooves is patterned by etching, thus forming the rounded collector stripes 632. The metal deposited on the opposite surface of the dielectric 620 is patterned by etching to create sharp edges, thus forming the emitter stripes 612.

The resulting collector stripes 632 have cross sections without corners or, at least the surfaces facing the emitter are rounded. In contrast, the emitter stripes 612 are formed with edges. In one approach, standard lithography is used to pattern the emitter stripes 612 on the dielectric substrate. The resulting cross section is typically rectangular or trapezoidal, with corners. The corners preferably have a radius of curvature not greater than 30 um.

Small ionic air flow generators may be fabricated using this approach. Any number of collector stripes and emitter stripes may be used. The device typically will have fewer emitter stripes, possibly only one, and they may be longer than the collector stripes. The pitch between emitter stripes typically may be 1-2 mm. There may be a large number of collector stripes that are spaced fairly close together, since the collector stripes play the role of a plane (with holes for air flow) in a line-plane geometry. The pitch between collector stripes may be less than a mm, for example 0.6 mm. The air flow area may be on the order of a few mm by a few mm, and the overall device size may be not much larger.

FIG. 7A are plan views of a collector conductor 730, dielectric 720, and emitter conductor 710, respectively, for a device with one emitter stripe and five collector stripes. The cross-sectional air flow area of this device is approximately 3 mm×5 mm, and the size of the entire device is approximately 5 mm×10 mm×2 mm thick. In FIG. 7A, the collector conductor 730 has five collector stripes 732. Each stripe 732 ends in patches 733 on both ends. These patches 733 are wider than the stripes and provide more support, stability and adhesion to the underlying substrate. The patches 733 are electrically connected to each other and to contact area 731, which provides electrical connection to the collector. The footprint of the entire device is shown by the dashed line 740.

The emitter conductor 710 has one emitter stripe 712, which is supported on opposite ends by patches 713 and 711. In this design, one of the patches is also the contact area 711 to make electrical connection to the emitter.

The dielectric substrate 720 has a single aperture 725 that covers the entire cross-sectional flow area. In this example, the dielectric 720 encloses the aperture 725, thereby creating a flow area between the emitter and the collector for the flow of air. The aperture 725 includes isolation notches 727, which increase the creep distance between the emitter and collector. If the aperture 725 were a rectangle without the notches 727, the creep distance would be shorter. The dielectric 720 may also have a cutout above the electrode 711, so that both electrodes 731 and 711 may be accessed from the same side.

FIG. 7B shows different creep distance paths between the collector 730 and emitter 710. Path 751 is from the collector end cap 733 to the emitter adhesion pad 713. Path 752 is from the collector electrical trace to the emitter adhesion pad 713. The creep distance along both of these paths are increased by the isolation notches 727. The creep distance along path 752 may also be mitigated by covering the collector electrical connection with a dielectric after the leads are attached. Path 753 is from the collector electrode 731 to the emitter adhesion pad 713. This creep distance may also be mitigated by coating the collector electrical connection with a dielectric after production.

In the designs described above, the collector is fabricated on one side of the dielectric substrate and the emitter is fabricated on the other side. In other designs, only one of the electrodes, either emitter or collector, may be fabricated on the dielectric. This is then combined with another subassembly that carries the other electrode.

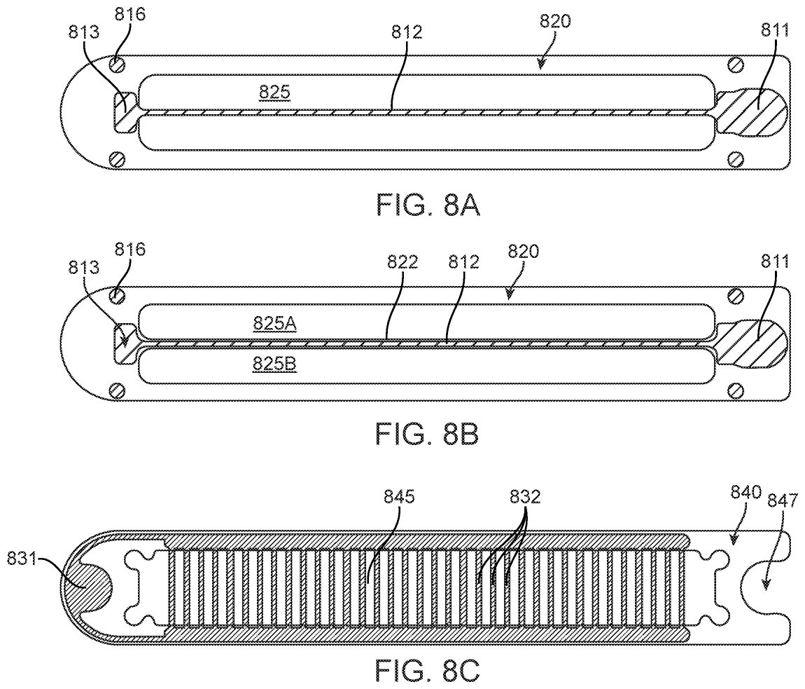

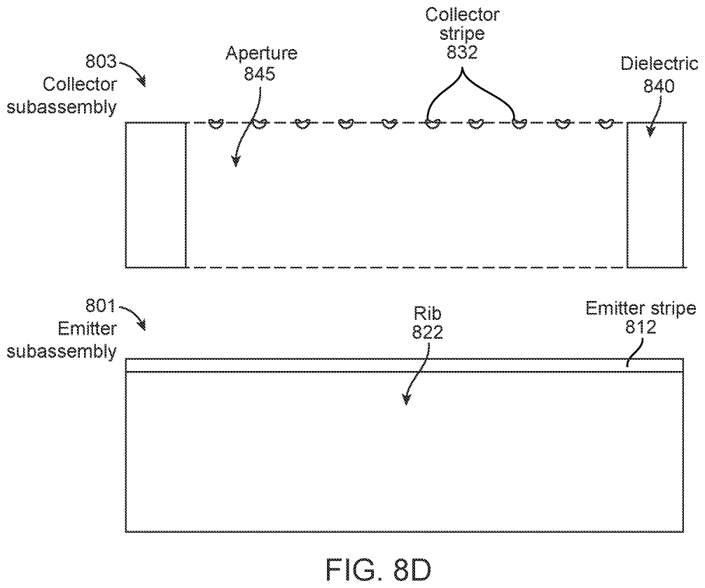

FIGS. 8A and 8B show examples where an emitter is fabricated on a dielectric substrate. FIG. 8A is similar to the designs shown in FIGS. 6-7, except there is no collector conductor. As in the above devices, the emitter stripe 812 is suspended over the aperture 825 in the dielectric 820. The stripe 812 is supported on both sides by adhesion pads 813 and 811, which on the one end also serves as a contact area 811 to make electrical connection to the emitter. Because the other subassembly may be mounted on top of this one, this device also includes leveling pads 816 to ensure level points of contact and to avoid unwanted contact with the long, skinny emitter stripe 812.

The emitter stripe 812 in FIG. 8A is long and skinny. FIG. 8B shows a design with additional support for the emitter stripe 812. The device shown in FIG. 8B has the same features as in FIG. 8A, except that the emitter stripe 812 is not suspended over a void. Instead, some dielectric is left to form a rib 822 that supports the emitter stripe 812. For example, the stripe may be 100 um wide and the supporting rib 822 may be 200 um wide. The rib 822 subdivides the aperture into two subapertures 825A, 825B.

FIG. 8C shows a corresponding collector subassembly, which may be used with the emitter subassemblies of either FIG. 8A or 8B. FIG. 8C is similar to the designs shown in FIGS. 6-7, except there is no emitter conductor. The collector stripes 832 are suspended over the aperture 845 in the dielectric 840. The stripes 832 are supported on both sides by adhesion pads, which connect to a contact area 831 for electrical connection. A notch 847 is cut out of the dielectric to allow access to the underlying emitter electrode 811.

FIG. 8D shows an exploded view of an ionic air flow generator assembled using the emitter subassembly 801 of FIG. 8B and the collector subassembly 803 of FIG. 8C. The cross section is taken through the emitter stripe 812 and rib 822, in a plane perpendicular to the collector stripes 832. In this example, the dielectric 840 from the collector subassembly serves as a spacer, but external spacers may also be used. The aperture 845 from the collector subassembly serves as the air gap between the emitter and collector.

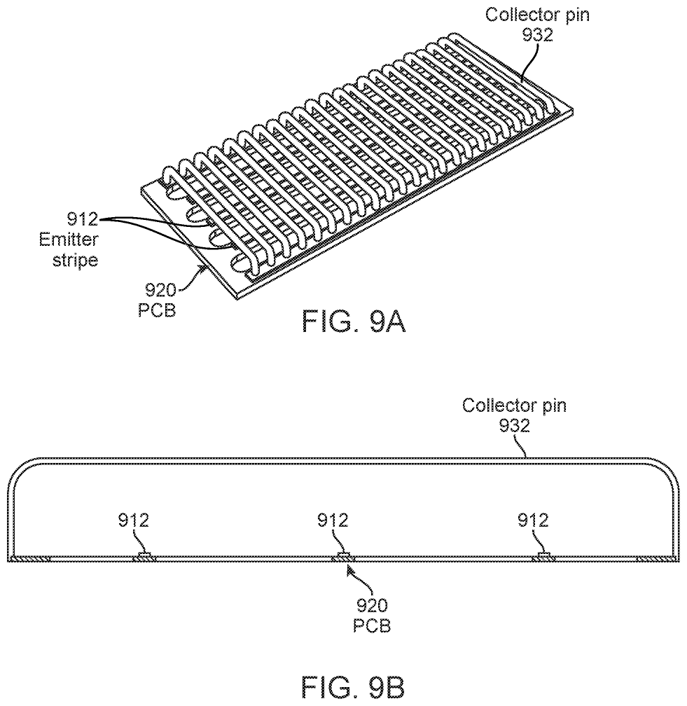

FIG. 9 shows yet another design, in which the emitter stripes are based on metal traces deposited on dielectric. FIG. 9A shows a perspective view of this ionic air flow generator, and FIG. 9B shows a cross-sectional view through a collector pin. In this examples, the emitter stripes 912 are metal traces deposited on a printed circuit board 920. The metal trace is shaped to concentrate the voltage and so form a corona. For example, the metal trace may have intentionally sharp corners. Also, the metal traces may be coated on the exposed side (i.e., side facing the collector) with different metals or metal alloys to enhance resistance to damage caused by occasional arcing, or otherwise increase the performance in generating a corona, for example nickel, palladium, gold, or platinum. The collector is a grid of pins 932 assembled in a cage structure. Alternatively, the collector may be a perforated plate.

The approaches described above also allow flexibility in control of the air flow generator. A controller may adjust the voltage applied to different emitter elements (ridges, stripes) and/or change which emitter elements are used. For example, extra emitter elements may be formed for redundancy. The controller switches to redundant emitter elements if other emitter elements fail, or a fuse may be incorporated into the material which can selectively, electrically isolate that element from the system. Alternatively, the number of emitter elements used may be adjusted to increase or decrease the air flow.

In addition, because these devices can be made with a short air gap, for example less than 2 mm or even less than 1 mm, lower operating voltages may be used. In some cases, the applied voltage is adjustable over a 500V-4 kV range, or over a 2-4 kV range. In other cases, the applied voltage may be less than 2 kV.

Although the detailed description contains many specifics, these should not be construed as limiting the scope of the invention but merely as illustrating different examples. It should be appreciated that the scope of the disclosure includes other embodiments not discussed in detail above. For example, embodiments of a similar structure may include two substrates with respective conductors created separately, and joined together as a subsequent step, or constructed such that air flow is routed in a lateral direction across the surface of the dielectric substrate rather than through perforations in it or the applied conductors. Various other modifications, changes and variations which will be apparent to those skilled in the art may be made in the arrangement, operation and details of the method and apparatus disclosed herein without departing from the spirit and scope as defined in the appended claims. Therefore, the scope of the invention should be determined by the appended claims and their legal equivalents.

What is claimed is:

1. An ionic air flow generator comprising:
a dielectric substrate having a first side and an opposing second side and an aperture through the dielectric substrate;
a first conductor comprising an emitter with one or more emitter stripes, wherein each emitter stripe is suspended across the aperture in the dielectric substrate and has two ends deposited on and supported by the first side of the dielectric substrate; and
a second conductor comprising a collector with multiple collector stripes, wherein each collector stripe is suspended across the aperture in the dielectric substrate and has two ends deposited on and supported by the opposing second side of the dielectric substrate;
wherein the dielectric substrate maintains an air gap between the emitter and collector, and a voltage applied to the emitter ionizes air at the emitter and the ionized air is drawn to the collector thereby creating a flow of air through the air gap.

2. The ionic air flow generator of claim 1 wherein the dielectric substrate comprises a ceramic substrate or a glass substrate.

3. The ionic air flow generator of claim 1 wherein the aperture is created by removing dielectric substrate, and the emitter and collector are formed by depositing the first and second conductors on the dielectric substrate before creating the aperture.

4. The ionic air flow generator of claim 1 wherein the emitter stripes and collector stripes form a regular pattern.

5. The ionic air flow generator of claim 1 wherein the emitter stripes have cross sections with corners.

6. The ionic air flow generator of claim 5 wherein at least one corner has a radius of curvature not greater than 30 um.

7. The ionic air flow generator of claim 1 wherein the collector stripes have cross sections without corners.

8. The ionic air flow generator of claim 1 wherein the emitter stripes are oriented perpendicular to the collector stripes.

9. The ionic air flow generator of claim 8 wherein:
the ends of the emitter stripes comprise patches that are deposited on and supported by the first side of the dielectric substrate on opposite sides of the aperture, the patches on each side of the aperture are electrically connected to each other and to an emitter electrode; and
the ends of the collector stripes comprise patches that are deposited on and supported by the second side of the dielectric substrate on opposite sides of the aperture, the patches on each side of the aperture are electrically connected to each other and to a collector electrode.

10. The ionic air flow generator of claim 8 wherein corners of the aperture include isolation notches that increase a creep distance between the emitter and collector.

11. The ionic air flow generator of claim 1 wherein the dielectric substrate maintains a consistent spacing for the air gap between the emitter and collector.

12. The ionic air flow generator of claim 1 wherein the dielectric substrate encloses the aperture, thereby creating a flow area between the emitter and the collector for the flow of air.

13. The ionic air flow generator of claim 1 wherein a flow area between the emitter and the collector for the flow of air is not more than 50 mm².

14. The ionic air flow generator of claim 1 wherein the flow of air is not less than 3 liters per minute per cm² of flow area.

15. The ionic air flow generator of claim 1 wherein the air gap between the emitter and the collector is not more than 2 mm.

16. An air flow system comprising:
an ionic air flow generator comprising:
a dielectric substrate having a first side and an opposing second side and an aperture through the dielectric substrate;
a first conductor comprising an emitter with one or more emitter stripes, wherein each emitter stripe is suspended across the aperture in the dielectric substrate and has two ends deposited on and supported by the first side of the dielectric substrate; and
a second conductor comprising a collector with multiple collector stripes, wherein each collector stripe is suspended across the aperture in the dielectric substrate and has two ends deposited on and supported by the opposing second side of the dielectric substrate;
wherein the dielectric substrate maintains an air gap between the emitter and collector, and a voltage applied to the emitter ionizes air at the emitter and the ionized air is drawn to the collector thereby creating a flow of air through the air gap; and
a controller that applies a voltage across the emitter and collector, wherein the applied voltage ionizes air at the emitter and the ionized air is drawn to the collector thereby creating a flow of air through the air gap.

17. The air flow system of claim 16 wherein the emitter comprises a plurality of emitter elements, and the controller is adjustable to apply the voltage to different ones of the emitter elements.

18. The air flow system of claim 16 wherein the emitter comprises a plurality of emitter elements, at least one of the emitter elements is redundant, and the controller applies the voltage to the redundant emitter element upon failure of another emitter element.

19. The air flow system of claim 16 wherein the controller applies the voltage to a different number of emitter elements based upon a desired rate of air flow.

20. The air flow system of claim 16 wherein the applied voltage does not exceed 2 kV.
